(19) United States
(12) Patent Application Publication
(10) Pub. No.: US 2016/0281128 A1
(43) Pub. Date: Sep. 29, 2016

(54) METHOD AND APPARATUS FOR DETECTING VECTOR-BORNE DISEASES IN MAMMALS

(71) Applicant: VETERINARY DIAGNOSTICS INSTITUTE, INC., Simi Valley, CA (US)

(72) Inventor: RANDY RINGOLD, West Hills, CA (US)

(73) Assignee: VETERINARY DIAGNOSTICS INSTITUTE, INC., Simi Valley, CA (US)

(21) Appl. No.: 15/034,860

(22) PCT Filed: Nov. 7, 2014
(86) PCT No.: PCT/US14/64453

§ 371 Date: May 5, 2016

Related U.S. Application Data
(60) Provisional application No. 61/902,058, filed on Nov. 8, 2013.

(51) Int. Cl. C12Q 1/48; G01N 33/68

(52) U.S. Cl. CPC C12Q 1/485; G01N 33/6893; G01N 2333/4737; G01N 2333/9122

(57) ABSTRACT

Canine subjects are screened for vector-borne diseases using Thymidine kinase (TK1) activity level alone or in conjunction with c-reactive protein (CRP) as biomarkers in the blood serum. While the canine subject may or may not display health symptoms indicative specifically of a vector-borne disease, the activity level of TK1 or in conjunction with the concentration of CRP are combined in a novel method that provides a practitioner the means of determining whether the subjects has a high probability of being affected by a vector-borne disease.

### CROSS-REFERENCES TO RELATED APPLICATIONS

The present application is a national phase US application of PCT application number PCT/US14/64453 filed on Nov. 14, 2014, which claims priority to U.S. provisional patent application No. 61/902,058 filed on Nov. 8, 2013

### FIELD OF THE INVENTION

The invention relates to a method and apparatus for detecting vector-borne diseases. More specifically, the invention comprises a method and apparatus for diagnosing the presence of vector borne diseases in a mammalian subject using the measurement of one or more biomarkers.

### BACKGROUND OF THE INVENTION

Vector-borne diseases (VBD) is a category of disease where an infectious micro-organism (a pathogen) is generally carried by a vector and transmitted to other bodies through the vector's natural behavior such as blood-sucking activity. Arthropods are the vectors for many disease-causing micro-organisms which are inoculated into a victim's body by sting and/or feeding on the victim's body. The most common arthropods that serve as vectors, in the case of humans and house pets or farm animals include blood sucking insects, such as mosquitoes, fleas, lice and other biting insects, and blood sucking arachnids, such as mites and ticks.

Typically, vectors become infected by a disease-causing microbe while feeding on infected vertebrates (e.g., birds, rodents, other larger animals, or humans). The microbe is then transmitted to other animals. In almost all cases, an infectious microbe must infect and multiply inside the arthropod before the arthropod is able to transmit the microbe, e.g., through its salivary glands.

Vector-borne pathogens have evolved unique mechanisms to persist/multiply within a host. Pathogens may have lost cell membrane Lipopolysaccharide (LPS) and peptidoglycan, which would otherwise activate innate immune defense mechanisms of the host. Pathogens may manipulate the vector's target neutrophil designed to destroy the pathogen or prevent the establishment of infection in a rather benign erythrocyte. Pathogens may suppress innate and adaptive immune responses to favor pathogen's survival, and/or express extensive antigenic variation in immunodominant surface proteins to permit evasion the immune response.

Vector-borne diseases represent a varied and complex group of diseases, which include known diseases such as anaplasmosis, babesiosis, bartonellosis, borreliosis (Lyme disease), dirofilariosis, ehrlichiosis, leishmaniosis, rickettsiosis and thelaziosis, however, new syndromes are still being uncovered. Vector-borne pathogens typically infect portions of the hematopoietic system, such as red blood cells, T-cell, monocytes, or granulocytes. The pathogen uses the host cell to replicate. The pathogen may remain within the hematopoietic system or transmit through the bloodstream to invade other cell lines within specific organs, such as the liver.

Without treatment, Vector-borne diseases are often characterized by three stages: 1) acute phase, 2) sub-clinical phase and 3) chronic phase. In humans, for example, the acute phase begins within 8-20 days following transmission and lasts for several weeks, and may be manifested by fever, depression, and weight loss. The subclinical phase may last from several months to years in which the host remains persistently infected without showing clinical signs. The last stage, chronic phase, resembles the first phase, but hemorrhaging or edema, and in severe cases death, may occur.

Many of the vector-borne diseases can cause serious (or even life-threatening) clinical conditions. Concerning dogs and cats, a number of these diseases carried by the latter two species may have zoonotic potential, i.e. potentially transmitted to humans (see table 1).

TABLE 1

| Disease | Vector |
|---|---|
| Leishmaniosis | Sand fly |
| Borreliosis | Tick |
| Bartonellosis | Flea |
| | Tick |
| Ehrlichiosis | Tick |
| Rickettsiosis | Tick |
| | Flea |
| Anaplasmosis | Tick |
| Dilofilariosis | Mosquito |
| Yersiniosis | Flea |
| Tularaemia | Tick |
| Coxiellosis | Tick |
| Tick-borne encephalitis | Tick |
| Louping ill | Tick |
| West Nile virus encephalitis | Mosquito |
| Trypanosomiosis | Triatoma bugs |

The incidence of vector-borne diseases in both humans and animals is increasing. Today, vector-borne diseases pose a growing global threat as they continue their spread far from their traditional geographical and temporal restraints as a result of changes in both climatic conditions and humans and pets travel patterns, exposing new populations to previously unknown infectious agents and posing unprecedented challenges to the medical community and veterinarians.

The diagnosis of vector-borne pathogens may be challenging, as clinical signs are frequently non-specific. Serological assays designed to detect the presence of antibodies to the pathogen frequently yield false negative results due to the immunosuppressive capability of the pathogen, which prevents the production of antibodies. False-positive serological results may be obtained from patients that have previously had a vector infection and still retain antibodies to the pathogen. The use of direct antigen detection with polymerase chain reaction (PCR) has a high degree of accuracy due to the direct detection of the pathogen however may also have false-negatives due to the transient presence of the pathogen in the blood stream at time of sampling.

Since early detection of VBD plays such a crucial role in the success of the treatment and the spread of the disease, there is a need for cost effective and least invasive screening methods that identify subjects with a VBD. Those patients that are screened as positive may undergo further diagnostic workup to identify the infecting pathogen and devise appropriate treatment.

### SUMMARY OF THE INVENTION

The invention provides a method and system that enable a practitioner to screen for a vector-borne disease in human or other mammalian subjects using one or more biomarkers. Whether symptoms indicative of a disease are (or are not) already displayed by the subject, an implementation of the invention enables the practitioner to reveal the presence of a vector-disease, which may lead to further diagnoses.

The invention utilizes any number of biomarkers that are indicative of dysregulated proliferation, such as Thymidine kinase type1. Furthermore, the invention may utilize any of the acute-phase proteins as a bio-marker. The increase or the decrease in the concentration of any number of APPs may be used to establish the suppression of an inflammatory response by a vector-borne pathogen.

In canine, the invention provides a screening method for vector-borne diseases using thymidine kinase type 1 (TK1) alone or in conjunction with c-reactive protein (CRP). The invention provides a method of computing a vector-borne disease index, which enables different practitioners to compare the results from different subjects and from different institutions. The latter index is obtained by first computing the product of the measurement of each biomarker and a corresponding weighing coefficient, the product is then digitized according to a discretization map, then the vector-borne disease index (VBI) is computed by summing the discretization value over all biomarkers. The discretization maps are optimally set such that an index value greater that one (1) is indicative of a high probability of the presence of VBD and should be considered for further diagnoses.

### BRIEF DESCRIPTION OF THE DRAWINGS

FIG. 1 is a flowchart diagram representing steps involved in developing a method for detecting and/or differentiating the presence of vector-borne diseases, in accordance with an embodiment of the invention.

FIG. 2A is a flowchart representing method steps involved in using a set of biomarkers in a diagnosis of one or more health statuses, in accordance with an implementation of the invention.

FIG. 2B is a graphical representation of a continuous index scale and defined index ranges corresponding to health statuses as taught by the invention.

FIG. 3 shows box and whisker plot representing statistical data for TK1 and CRP for a group of canine subjects carrying VBD and a group of healthy subjects.

FIG. 4 is a plot showing the relationship between the sensitivity and the specificity of the VBD index as computed above from the data. Plot 410 shows a curve 420 that plots the sensitivity of the vector-borne disease index (VBI) as a function of the specificity for range of cutoff values.

FIG. 5 is a plot showing the relationship between the sensitivity and the specificity of the latter index as computed using the values of Table 5.

FIG. 6 is a plot showing the relationship between the sensitivity and the specificity of index computation using method 1 and method 2 (see above), and TK1 and CRP individually.

FIG. 7A shows plots of the sensitivity 710 and specificity 720 as a function of the cutoff value, using TK1 activity level as input data.

FIG. 7B shows plots of the sensitivity 750 and specificity 760 as a function of the cutoff value, using the concentration of CRP as input data.

FIG. 8 is a plot showing the relationship between the sensitivity and the specificity of index computation using cutoff values in a range of values for both TK1 and CRP.

### DETAILED DESCRIPTION OF THE INVENTION

The invention is a method and apparatus by which a practitioner determines whether a human or another mammalian may be affected by a vector-borne disease (VBD) by measuring the presence of one or more biomarkers and computing an index that provides the likelihood of the presence of vector-borne pathogen.

In the following description, numerous specific details are set forth to provide a more thorough description of the invention. It will be apparent, however, to one skilled in the pertinent art, that the invention may be practiced without these specific details. In other instances, well known features have not been described in detail so as not to obscure the invention. The claims following this description are what define the metes and bounds of the invention.

The present disclosure shares some aspects of the concepts and the methods described in U.S. patent application Ser. No. 13/672,649, Ser. No. 13/672,677 and Ser. No. 13/672,687, International patent application No. PCT/US12/23135 and U.S. patent application Ser. No. 14/372,328, each of which is included by reference in its entirety in the present disclosure.

### TERMINOLOGY

Abbreviations “TK” and TK1, as used in the disclosure, interchangeably refer to thymidine kinase type 1. Thymidine kinase as a biomarker may be measured using its enzymatic activity as a marker for its presence, for example, in the blood. The activity level is usually provided as Unit per volume of blood. The scope of the invention encompasses however all available means for determining the amount of TK1 in the blood.

Throughout the description, the terms individual, subject or patient may refer to an animal subject or a person whose biological data are used to develop and/or use an implementation of the invention. The subject may be normal (or disease-free) or showing any combination (e.g., including absence of) symptoms.

The term biomarker refers to any indicator in any body part (e.g., bodily fluid or tissue) that may be collected and the presence of a biomarker measured through any of its manifestations such as enzymatic activity, mass, concentration, cell count, cell shrinkage/shape, deoxyribonucleic acid (DNA) and/or ribonucleic acid (RNA) genetic level of expression or any aspect of the biochemical or the physiological markers that may be related to one or more health conditions. Moreover, for the purpose of designing health status indices (see below) a biomarker data may be any related data that may be considered for diagnosing a disease (or the probability of occurrence thereof) such as age, sex, any biometric data, genetic history (e.g., parent's health status or presence of any affection in the family) or any other data that may contribute to the diagnosis of a disease.

In the disclosure the measurement of biomarkers are typically concerned with measuring the concentration (or the activity level) of the biomarker in the blood serum. One with ordinary skills in the pertinent art would recognize that the invention may be practiced using other body fluids such as cerebrospinal fluid, lymph or any other body fluid for which the invention has been implemented. In addition, implementations of the invention may adequately select more than one body fluid for testing for each or any number of biomarkers considered in a test of detecting VBD.

The term “index” is used throughout the disclosure to refer to a dependent variable that is calculated using two or more data inputs such as the level of a biomarker in the blood stream. An index is computed with the goal of classifying subjects into groups based on disease status. For example, a subject that may be apparently healthy (e.g., showing no signs of VBD), but that has been diagnosed with VBD, would have an index value that reflects the health status, in accordance with embodiments of the invention.

The term “user” may be used to refer to a person, machine or a computer program acting as or on behalf of a person.

In using an enzyme as a biomarker, the level of activity of the enzyme may depend on the type of substrate in the test kit, in addition to other parameters such as temperature and pH. Thus, the disclosure considers any adjustments to the calculation/measurement of the enzymatic activity a practitioner may make to practice the invention as inherent steps required for specific implementations of the invention without deviating from the concept of the invention.

Diagnosing Vector-Borne Disease

The invention aims at providing cost-effective easy to implement screening for VBD. Therefore, an implementation for screening for VBD in accordance with the invention requires basic laboratory equipment for measuring proteins and/or enzymatic activity levels in body fluids, comprising body fluid collection kits (e.g., red top tubes, needles and syringes), body fluid storage and handling equipment, blood serum separation tools (e.g., centrifuges), test tubes and any other machine or tools for a laboratory test. The invention may be practiced using any available test kits for measuring any target biomarker for a specific implementation.

Inflammation is a process to defend against foreign invasion by activating a cascading sequence of events including the formation of antibodies. Vector-borne pathogens have evolved to suppress this inflammatory host response to the infection.

An inflammatory process leads to the activation of the cytokine network. In the early phase of this process, proinflammatory cytokines (TNF-α, IL-1β, INF-γ and IL-12) are released. The activity of proinflammatory cytokines is counteracted by the production of anti-inflammatory cytokines (IL-4, IL-10, IL-13 and TGF-β) and soluble inhibitors of proinflammatory cytokines (soluble TNF-α receptor, soluble IL-1 receptor, and IL-1 receptor antagonist).

In response to the formation of cytokines, a complex series of reactions are initiated called the acute-phase response (APR). These reactions aim to prevent ongoing tissue damage, isolate and destroy the infectious organism (if present) and activate the repair processes necessary to restore the host/organism's normal function. The acute-phase response is characterized by leukocytosis, fever, alterations in the metabolism of many organs as well as changes of the concentration of various acute-phase proteins (APPs) in the blood plasma.

Acute-phase proteins (APPs) have been defined as any protein the concentration of which in the plasma changes by at least twenty five percent (25%) during an inflammatory disorder. Those proteins the concentration of which increases are defined as positive acute-phase proteins (e.g., fibrinogen, serum amyloid A, albumin, C-reactive protein), and those proteins the concentration of which decreases are defined as negative acute-phase proteins (e.g., albumin, transferrin, insulin growth factor I).

For example, C-reactive protein (CRP) is a major APP and has been shown to be an effective measure of general inflammation. The concentration of CRP or any serum APP level correlates to both the severity and the duration of the inflammatory stimuli.

The invention utilizes any of the acute-phase proteins as a bio-marker. The increase or the decrease in the concentration of any number of APPs may be used to establish the suppression of an inflammatory by a vector-borne pathogen. Furthermore, the invention may utilize any number of biomarkers that are indicative of dysregulated proliferation, such as Thymidine kinase type1.

Thymidine kinase type 1 (TK1) is a salvage enzyme involved in the synthesis of DNA precursors. Thymidine kinase is expressed only in phase S though G2 of cell division (Mitosis). TK1 levels have been shown in numerous studies, both in humans and animals, to correlate with the proliferative activity of dysregulated replication, a hallmark of tumor disease. Serum TK1 concentrations have been studied in human and veterinary applications.

The study upon which the invention is based hypothesizes that TK1 may be elevated in situations where non-neoplastic dysregulated cellular division occurs leading to a false positive result. This may happen when a pathogen invades a host cell and uses cellular processes in the replication of the pathogen. As shown below, canine subjects infected by vector-borne pathogens have an increased TK1 concentration, presumably due to the pathogen's replication.

Embodiments of the invention may utilize the measure of TK1 activity in combination with measuring the concentration of one or more APPs, in order to evaluate the probability that a mammal is a carrier of VBD.

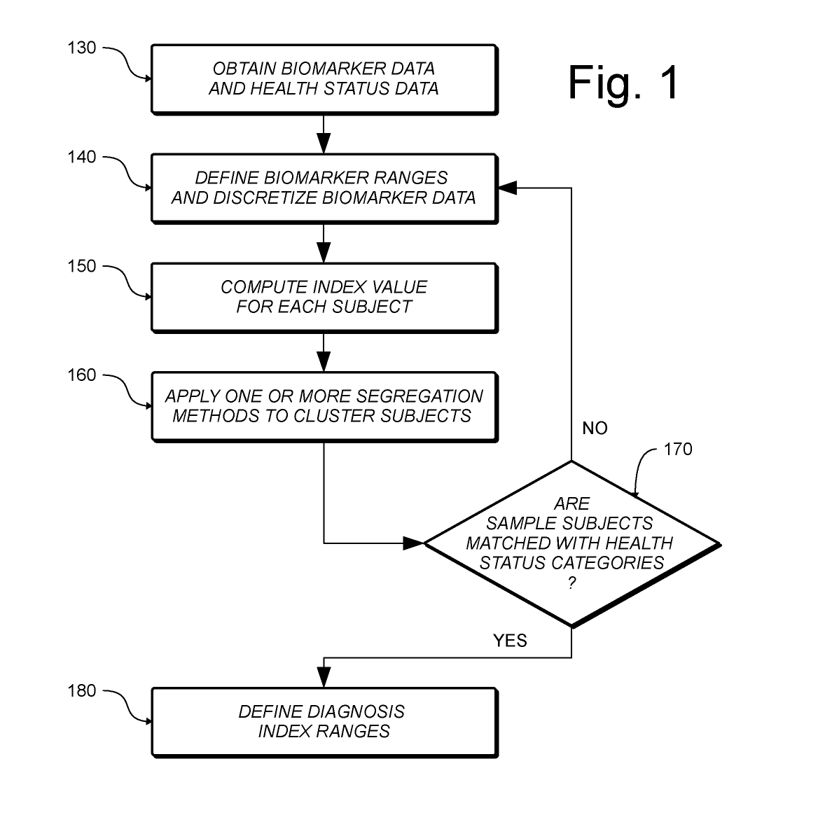

FIG. 1 is a flowchart diagram representing steps involved in developing a method for detecting and/or differentiating the presence of vector-borne diseases, in accordance with an embodiment of the invention.

Step 130 represents collecting data from a group of subjects. The group of subjects may be a sample of subjects comprising normal subjects (i.e. healthy) or unaffected by VBD, and affected subjects showing any level of severity of symptoms and/or other indicators. Bodily fluids, tissue or any other body sample may be appropriately collected in order to measure the level of each biomarker of the set of biomarkers, such as Thymidine kinase, C-reactive protein etc.

In addition, the subjects may undergo a plurality of tests, such as histological, radiological tests or any other test designed to establish the presence or absence of the target disease(s). Other tests may be conducted on each subject to either further confirm VBD or rule out other diseases that may share common symptoms with VBD.

Moreover, other non-disease related data may also be considered. The latter data comprise age, sex, any biometric data, genetic history (e.g., parent's health status or presence of any affection in the family) or any other data that may contribute to the diagnosis of a disease.

The level of each biomarker may be expressed in one or more unit types that characterizes the level of the presence of the biomarker in the body fluid/tissue under consideration. Thus, an enzyme may be characterized by the level of its enzymatic activity, a protein, a hormone or any other biomarker may be expressed by a concentration level such as its mass or moles per volume of tissue or bodily fluid.

Step 140 represents the process of defining range values for each biomarker, and involves discretizing the data, which comprises attributing a score number to each previously defined range of a biomarker level. For example the level of thymidine kinase may be represented by three ranges, the first range may be attributed the value zero (0), the second range may be attributed the value one (1) and the third range may be attributed the value two (2).

Step 150 represents computing an index value for each subject as follows:

$$I = {\sum }_{i = 1}^{i = N} {C}_{i} \cdot {L}_{i} \qquad ( 1 )$$

where the index value “I” for each subject may be the sum of the product of the score level “L” (e.g., computed at step 140) and a coefficient “C” associated with the “i^{th}” data input for a number “N” of data inputs (e.g., biomarker level, age, biometric data etc.). The coefficient “C” may be determined empirically as shown below at steps 160 and 170.

Step 160 represents applying one or more methods for segregating subjects using the health status data and the computed index values. For example, the method of segregation may be the Receiver Operating Characteristic (ROC) curve analysis. ROC curve analysis is a well known method in the medical field for determining whether a correlation between the level of a biomarker may serve as an indicator of the presence of a health condition. The latter is possible for example when there is a strong correlation between the amount of a substance in the body (e.g., high cholesterol) and a health condition (e.g., sclerosis of blood vessels).

Using the ROC curve analysis on the index values of all subjects in the group, it is possible to determine whether there is a cutoff value capable of classifying individuals into groups matching their health status. For example, if subjects carrying a disease are labeled as positive and the non-carriers are labeled as negative, the ROC curve analysis may yield a threshold that classifies the subjects into an above and a below-threshold groups matching the health statuses carrier and non-carrier of the disease, respectively. There may be false positives and false negatives for each chosen cutoff value in the range of possible values. The rate of success in determining true positive cases is called “Sensitivity”, whereas the rate of success in determining true negative cases is called “Specificity”. Sensitivity and specificity for a plurality of cutoff values are computed. Sensitivity and Specificity are rates, and thus may be expressed in the range of zero (0) to one (1), or as a percentage from zero (0) to one hundred percent (100%). The results are plotted as Sensitivity values versus one (1) (or 100% depending on the unit of choice) minus the corresponding specificity. The area under the curve (AUC) reveals whether ROC analysis may be a valid classifier of the data: the closer the AUC is to 100%, the better classifier is the ROC analysis. On the contrary, the ROC analysis may not be considered for classification purposes if the AUC is closer to 50%, which is considered close to a random process. In general, the ROC method of analysis may be considered valid, if the AUC is at least 0.8.

Moreover, each threshold value yields a “Sensitivity” and “Specificity”. In populations where where ROC analysis appears adequate, the “Sensitivity” curve decreases as the “Specificity” increases. At a particular threshold, the apex, the total of Sensitivity and Specificity is at a maximum. The apex is typically chosen as the threshold of classification if it yields a Sensitivity and Specificity each above 0.85, otherwise a threshold for Specificity and a threshold for Sensitivity may be respectively selected to yield a success rate of at least 0.85.

ROC analysis is one of any existing methods that may be utilized in embodiments of the invention to detect clusters in the data that define the clustering boundaries capable of segregating subjects into groups matching health status categories. For example, k-means clustering, hierarchical clustering, neural networks or any other clustering clustering method may be utilized in one or more embodiments of the invention. Furthermore, an embodiment of the invention may conduct the steps of FIG. 1 using a plurality of methods of clustering the data to achieve the results of the invention. The final clustering method that may be retained in any particular embodiment of the invention may be the one that yields the highest success rate of the diagnosis.

Step 170 represents computing success scores of the method of segregating of subjects in the test group. If the success level of the segregation into health categories is not satisfactory (e.g., no statistical difference compared to a population drawn from a random process), the parameters for computing the index values are revised and the analysis is repeated at step 140. The process of searching for optimal parameters may be repeated until the result of classification of subjects reaches (or exceeds) an acceptable success rate. Otherwise, if no optimal parameters may be found, the result may indicate that the chosen set of biomarkers is unsuitable for segregating the subjects, based on the index method in question, into the proposed health status categories.

The search for optimal parameters may involve changing one or more boundary values for discretizing biomarker values, and/or the weight coefficients associated with each biomarker in computing the index value for each subject. The search method may be manual i.e. an expert practitioner may set the initial parameters and adjust them, through multiple iterations of computation, while considering the outcome of the success rate of classification of subjects into health status categories. Implementations of the invention may also use numerical methods for automatic search to optimize parameters. Such methods comprise brute force search, where a large number of values of parameters and combinations thereof are tested. The numerical methods for determining optimal values may use gradient descent search, random walk search or any other mathematical method for searching for optimal parameters in order to achieve the goal of maximizing the success rate of the classification of subjects into correct corresponding health status categories.

Computer programs for conducting a search, in accordance with an implementation of the invention, require ordinary skills in the art of computer programming. Moreover, existing computer programs may be adapted (through a programming scripting language) to carry out a search process in an implementation of the invention. Computer programs include such programs as Mathematica™, Matlab™, Medcalc™, or any other available computer program may be used.

Step 180 represent the final step of determining the final parameters (or range thereof) that may be used in a diagnosis of the target disease(s). The optimal parameters include the coefficient associated with each biomarker, the number of ranges and the boundary values that define the ranges for each biomarker. Step 180 also includes determining the index range boundaries that define the categories as defined by the health status of subjects. The latter parameters may be used in systems for diagnosing whether a subject is a carrier of the a disease, as will detailed below in the method of use.

The invention provides a means for facilitating the display and read out of the results by defining the boundaries between ranges as discrete values for ease of use. For example, a scale comprising two health statuses, such as “disease present” and “disease not present”, may be defined has having a discrete boundary, such as one “1”, where the scale range lower than “1” may be mapped to “disease not present” status, while the scale range greater than “1” is mapped to “disease present” status.

Defining range boundaries as discrete values may be carried out during the search for the optimal parameters (as described above). The discrete range boundary values may also be provided computationally (e.g., using multipliers and offsets) subsequent to determining the optimal parameters.

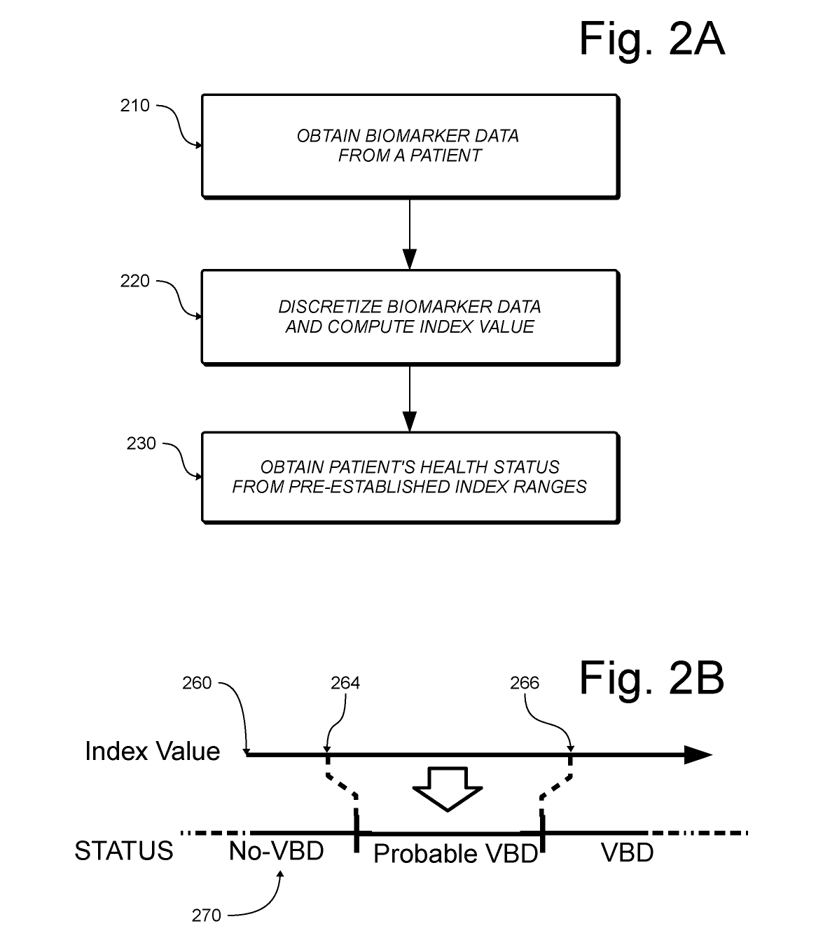

FIG. 2A is a flowchart representing method steps involved in using a set of biomarkers in a diagnosis of one or more health statuses, in accordance with an implementation of the invention. Provided a set of pre-established optimal parameters that yield an acceptable success rate for classifying subjects into health categories based on a computed index from biomarkers, the invention provides a method and system for testing whether a new patient is likely a carrier of a suspected disease using biomarkers. Step 210 represents obtaining data from a patient. Similarly to step 130 and depending on the specific set of biomarkers involved in a diagnosis, bodily fluids, tissue and any other data necessary for the diagnosis are collected and the level of each biomarker is assessed.

Step 220 represents computing an index value for the patient. Provided the discretization boundary values for each biomarker, the level of each biomarker is converted into a score value, and provided the coefficient associated with each biomarker, the index value for the patient may be computed using equation (1).

Step 230 represents determining a patient's health status group. The patient's computed index value is compared to that of the established boundary values for health status categories. As described above, the established mapping between index values allows for ascertaining the health condition of a patient using its own index value.

FIG. 2B is a graphical representation of a continuous index scale and defined index ranges corresponding to health statuses as taught by the invention. Line 260 represents a continuous scale of index values. Health status scale 270 represents the health status categories for which the diagnosis method was initially developed in accordance with the teachings of the invention. The health status scale may define two (2) or more health statuses, such as, in the case of cancer, non-carrier of a VBD, low to medium probability of carrying a VBD germ and high-probability of carrying a VBD germ. Index values 264 and 266 may define the boundaries to read out the health status of a patient in question. Thus, a patient's index value that is less than about boundary 264 would indicate the patient in question is in a first health status category, an index value greater than about boundary 264 and less than about boundary 266 would indicate the patient is in a second health category while an index value greater than boundary 266 would indicate that the patient is in a third health status category.

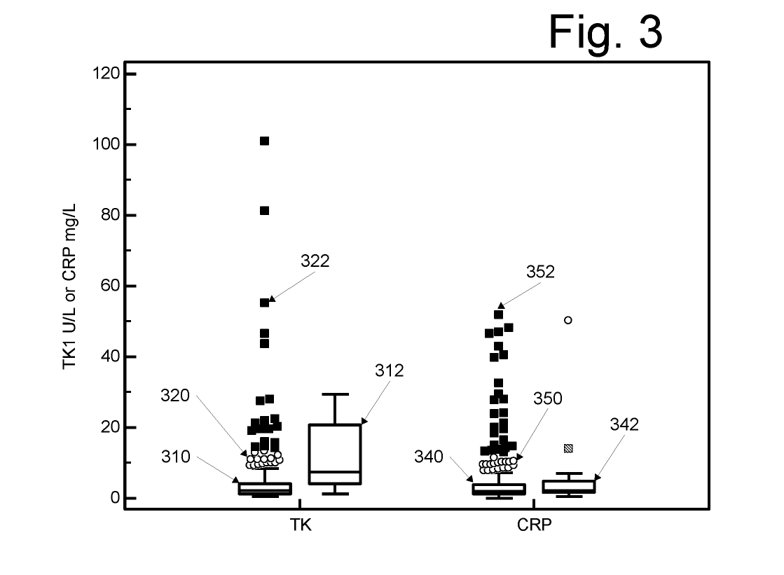

FIG. 3 shows box and whisker plot representing statistical data for TK1 and CRP for a group of canine subjects carrying VBD and a group of healthy subjects. Box and whisker plots 310 and 312 represent aggregate data of TK1 enzymatic activity in blood serum, respectively, for the healthy group and the VBD group. Box and whisker plots 340 and 342 represent aggregate CRP blood serum concentration, respectively, for the healthy group and the VBD group. Plot marks 320 and 322 represent TK1 enzymatic activity in blood serum for each individual subject, respectively, in the healthy group and the VBD group. Plots marks 350 and 352 represent CRP concentration in blood serum for each individual subject, respectively, in the healthy group and the VBD group.

Method 1.

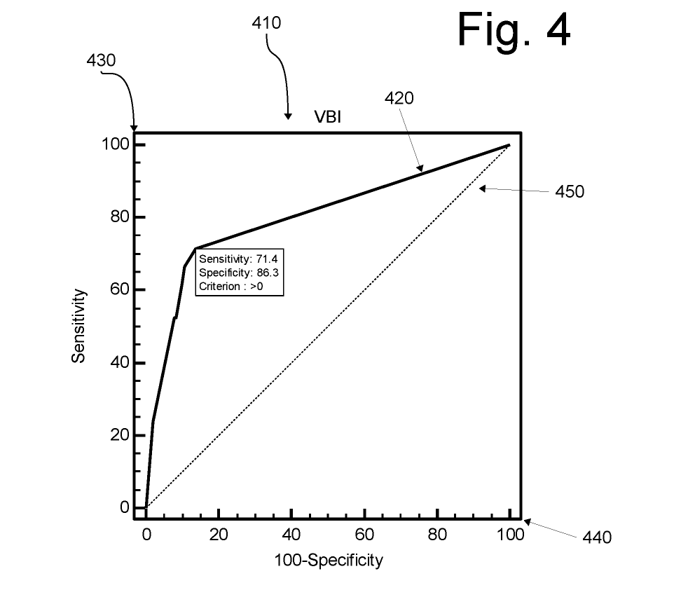

A diagnosis for VBD in canine has been developed, in accordance with the teachings of the invention, with a cohort of 386 patients. Statistical analysis results of the study as processed, using Receiver Operating Characteristic (ROC) curves, are presented in Table 2, Table 3 and FIG. 4.

TABLE 2

| | |
|---|---|
| Sample size | 386 |
| Positive group (i.e. Status 1) | 21 |
| Negative group (i.e. Status: 0) | 365 |
| Disease prevalence (%) | unknown |
| Area under the ROC curve (AUC) | 0.802 |
| Standard Error | 0.0600 |
| 95% Confidence interval (±1.96 SE) | 0.684 to 0.919 |
| z statistic | 5.034 |
| Significance level P (Area = 0.5) | <0.0001 |
| Youden index J | 0.5773 |
| Associated criterion | >0 |

Table 3 shows the data discretization and assigned values for ranges of thymidine kinase enzymatic activity levels and CRP concentrations.

TABLE 3

| TK1 (U/l) | CRP (mg/l) | Assigned |
|---|---|---|
| <6.5 | TK1 <6.5 or >=7 | 0 |
| >=6.5 and <=19.9 | >=4.1 and <=6.9 | 1 |
| >20 | <=4 | 2 |

An index VBI is then calculated using the digital assigned values dTK1v and dCRPv, respectively, obtained for TK1 and CRP as follows:

VBI=(dTK1v*1.8)+(dCRPv*3) (2)

FIG. 4 is a plot showing the relationship between the sensitivity and the specificity of the VBD index as computed above from the data. Plot 410 shows a curve 420 that plots the sensitivity of the vector-borne disease index (VBI) as a function of the specificity for range of cutoff values. The sensitivity scale 430 is expressed between “0” and “100”, “0” meaning that the chosen cutoff value provides a test that is not sensitive i.e. no subject is determined being a having VBD, and “100” meaning that the test positively determines all subjects have VBD. The specificity values 440 are expressed in 100 minus the measured specificity.

In FIG. 4, curve 450 (straight line) represents the relationship between the specificity and the sensitivity, in a Receiver Operating Characteristic (ROC) analysis of an inconclusive hypothetical test. FIG. 4 shows that the invention provides a test for which the specificity and sensitivity relationship represented by curve 420 rises toward the 100% sensitivity level and remains above curve 450.

Table 4 shows the ROC results:

TABLE 4

| Criterion | Sensitivity | 95% CI | Specificity | 95% CI | +LR | 95% CI | −LR | 95% CI |
|---|---|---|---|---|---|---|---|---|
| ≧0 | 100.00 | 83.9-100.0 | 0.00 | 0.0-1.0 | 1.00 | 1.0-1.0 | | |
| >0 | 71.43 | 47.8-88.7 | 86.30 | 82.3-89.7 | 5.21 | 3.6-7.6 | .33 | 0.2-0.7 |
| >1.8 | 66.67 | 43.0-85.4 | 89.32 | 85.7-92.3 | 6.24 | 4.1-9.5 | .37 | 0.2-0.7 |
| >3.6 | 61.90 | 38.4-81.9 | 90.14 | 86.6-93.0 | 6.28 | 4-9.9 | .42 | 0.2-0.7 |
| >4.8 | 52.38 | 29.8-74.3 | 91.78 | 88.5-94.4 | 6.37 | 3.7-10.9 | .52 | 0.3-0.8 |
| >6.6 | 52.38 | 29.8-74.3 | 92.05 | 88.8-94.6 | 6.59 | 3.9-11.3 | .52 | 0.3-0.8 |
| >7.8 | 23.81 | 8.2-47.2 | 98.08 | 96.1-99.2 | 12.41 | 4.3-35.8 | .78 | 0.6-1.0 |
| >9.6 | 0.00 | 0.0-16.1 | 100.00 | 99-100 | | | 1.0 | 1.0-1.0 |

Method 2.

In one embodiment of the invention, a simple index may be built whereby specimens are assigned a value on the basis of their TK1 level of activity and the concentration of CRP, namely, using an elevated level TK1 wherein the concentration of CRP is normal to diagnose VBD. Using the following cutoff values in Table 4.

TABLE 5

| TK1 (U/l) | CRP (mg/l) | Assigned |
|---|---|---|
| <6.5 | <=4 | 0 |
| >=6.5 | >=4.1 | 1 |

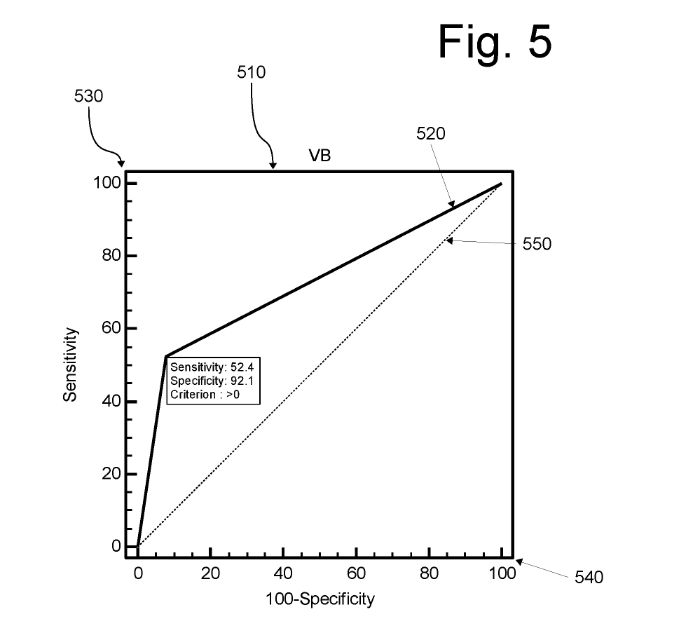

The results are shown in Table 6 and 7 and FIG. 5. FIG. 5 is a plot showing the relationship between the sensitivity and the specificity of the latter index as computed using the values of Table 5. Plot 510 shows a curve 520 that plots the sensitivity of the vector-borne disease index as a function of the specificity for range of cutoff values. The sensitivity scale 530 is expressed between “0” and “100”, “0” meaning that the chosen cutoff value provides a test that is not sensitive i.e. no subject is determined being as being affected with VBD, and “100” meaning that the test positively determines all subjects have VBD. The specificity values 540 are expressed in 100 minus the measured specificity. Curve 550 (straight line) represents the relationship between the specificity and the sensitivity, in a Receiver Operating Characteristic (ROC) analysis of an inconclusive hypothetical test.

TABLE 6

| | |
|---|---|
| Sample size | 386 |
| Positive group (i.e. Status 1) | 21 |
| Negative group (i.e. Status: 0) | 365 |
| Disease prevalence (%) | unknown |
| Area under the ROC curve (AUC) | 0.722 |
| Standard Error | 0.0685 |
| 95% Confidence interval (±1.96 SE) | 0.588 to 0.856 |
| z statistic | 3.242 |
| Significance level P (Area = 0.5) | 0.0012 |
| Youden index J | 0.4444 |
| Associated criterion | >0 |

Table 7 shows the ROC results using the latter method of selecting cutoff values to compute the index.

TABLE 7

| Criterion | Sensitivity | 95% CI | Specificity | 95% CI | +LR | 95% CI | −LR | 95% CI |
|---|---|---|---|---|---|---|---|---|
| ≧0 | 100.00 | 83.9-100.0 | 0 | 0-1 | 1.00 | 1.0-1.0 | | |
| >0 | 52.38 | 29.8-74.3 | 92.05 | 88.8-94.6 | 6.59 | 3.9-11.3 | 0.52 | 0.3-0.8 |
| >1 | 0.00 | 0.0-16.1 | 100 | 99-100 | | | 1.00 | 1.0-1.0 |

Thus, the invention determines, that when the VBI of a subject is determined to be above zero (0), the subject is likely a carrier of VBD.

A comparative study has been carried out in order to investigate the performance of the methods of the invention in predicting a positive diagnosis of VBD. Thus, the diagnosis performance was checked using the computation of indices based on the method 1 or method 2 above, or individually TK1 or CRP measurements.

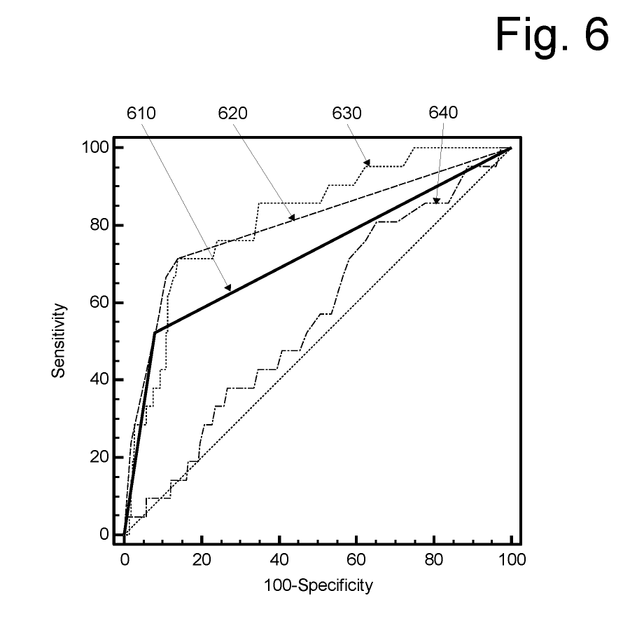

FIG. 6 is a plot showing the relationship between the sensitivity and the specificity of index computation using method 1 and method 2 (see above), and TK1 and CRP individually. Curve 610 shows the ROC results of using the index as disclosed in method 2, curve 620 shows the results obtained with using method 1, curve 630 shows the results obtained with using TK1 alone, and curve 640 shows the results obtained with using CRP alone.

Method 3.

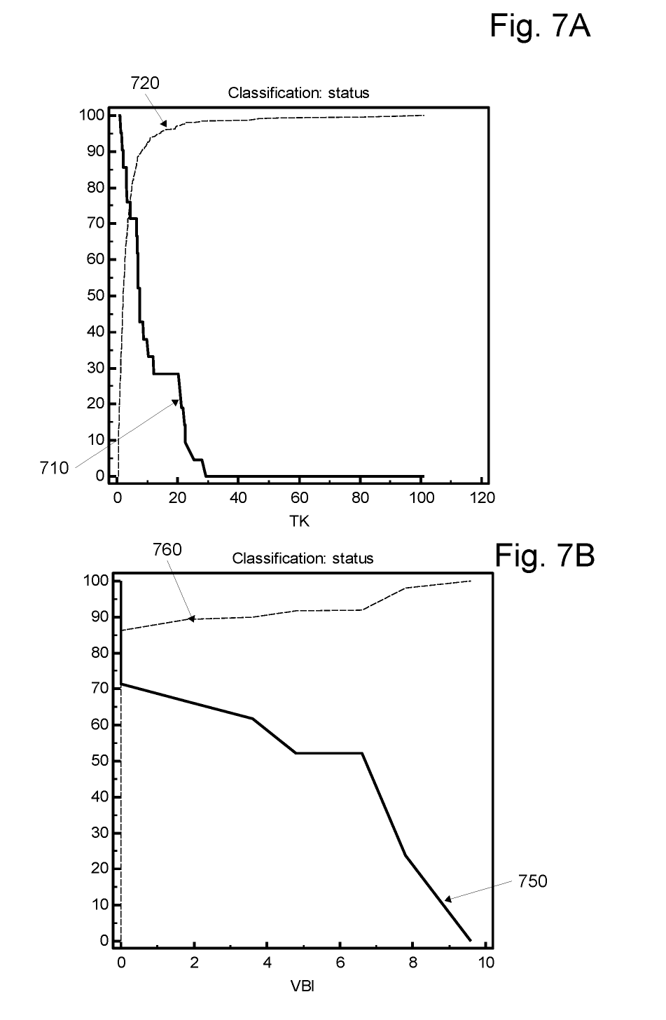

In one embodiment of the invention, a rapid diagnostic may be carried out using TK1 alone. In fact, as shown by curve 630 (in FIG. 6) using TK1 alone yields an ROC AUC of 0.817. The apex of curve 630, i.e. where accuracy is maximal (sensitivity of 0.71 and specificity of 0.86) as illustrated by curves 710 and 720 of FIG. 7A, is achieved using TK1 activity cutoff value of 6.5 U/L. The use of TK1 alone in apparently healthy dogs provides a fast screen for VBD, while overcoming the problem of lack of sero-conversion that affects antibody-based screening, and the problem of false negatives that affects PCR analyses that may reveal only a transient blood migration of the pathogen.

Using CRP alone yields curve 640, which has a ROC AUC of 0.558. The expected low AUC of curve 640 may be attributed to the lack of an immune response due to the masking of the pathogen to the host. In both method 1 and method 2, higher specificity (a desired outcome of the invention) is achieved than the use of TK1 alone. Method 1 has the benefit of maintaining a high ROC AUC and also maintains the same apex of the curve as TK1 alone. Patients that may have cancer (not included in this cohort) and an elevated TK1 will automatically be eliminated due to concurrent elevation in CRP (as shown in patent PCT/US12/23135). Patients that have just an inflammatory disease will only have elevated CRP concentration and will be eliminated as well.

FIG. 7A shows plots of the sensitivity 710 and specificity 720 as a function of the cutoff value, using TK1 activity level as input data. FIG. 7B shows plots of the sensitivity 750 and specificity 760 as a function of the cutoff value, using the concentration of CRP as input data.

Table 8 summarizes the results of the comparative study involving method 1, method 2, and individually TK1 and CRP. The study involved a cohort of 386 subjects, 21 subjects of which were carriers of VBD and 365 of which were healthy. The area under the curve (AUC) of the ROC analysis, the standard error (SE) and the 95% confidence interval are given for the different analyses using Method 1, method 2, TK1 and CRP as an input to verify which biomarker is a better classified of subjects that carry VBD versus healthy subjects.

TABLE 8

| | | | 95% CI (AUC ± 1.96 |
|---|---|---|---|
| | AUC | SE | SE) |
| Method 1 | 0.801 | 0.0600 | 0.684 to 0.919 |
| Method 2 | 0.722 | 0.0685 | 0.587 to 0.856 |
| Method 3 | 0.817 | 0.0461 | 0.727 to 0.907 |
| (TK1) | | | |
| CRP | 0.558 | 0.0616 | 0.437 to 0.678 |

Table 9 shows the results of pairwise comparison of the data between the four (4) methods under consideration. The difference between area under the curves (Diff. AUC), the stand error (SE), the 95% Confidence Interval (SE), z statistic (z Stat.), and the Significance level (Sig.) are considered for the comparison.

TABLE 9

| | Method | Method | Method | Method | Method | Method |
|---|---|---|---|---|---|---|
| | 1 vs. | 2 vs. | 2 vs. | 1 vs. | 1 vs. | 3 vs. |
| | Method 2 | Method 3 | CRP | Method 3 | CRP | CRP |
| Diff. | 0.0795 | 0.0952 | 0.164 | 0.0157 | 0.243 | 0.259 |
| AUC | | | | | | |
| SE | 0.0406 | 0.0640 | 0.0743 | 0.0465 | 0.0745 | 0.0705 |
| 95% | −0.0000288 | −0.0303 | 0.0184 | −0.0754 | 0.0975 | 0.121 |
| CI | to 0.159 | to 0.221 | to 0.309 | to 0.107 | to 0.389 | to 0.397 |
| z stat. | 1.959 | 1.487 | 2.208 | 0.338 | 3.269 | 3.675 |
| Sig., | 0.0493 | 0.1369 | 0.0272 | 0.7350 | 0.0011 | 0.0002 |
| P= | | | | | | |

To further characterize the methods of the invention and investigate the ranges within which the methods of the invention may be applicable, a non-exhaustive comparative study has been carried out. The outcome demonstrate that while some values for computing the index yield optimal results, with respect to positively diagnosing VBD subjects, the results show that other values e.g., within a statistical range of values, may yield acceptable diagnosis results. Therefore, each value disclosed herein should be interpreted as representing a range of values which are capable of yielding satisfactory diagnosis results. Furthermore, a practitioner using an embodiment of the invention may be provided the capability of selecting values that are different from the specific disclosed values for computing the index insofar as they are in the disclosed satisfactory ranges.

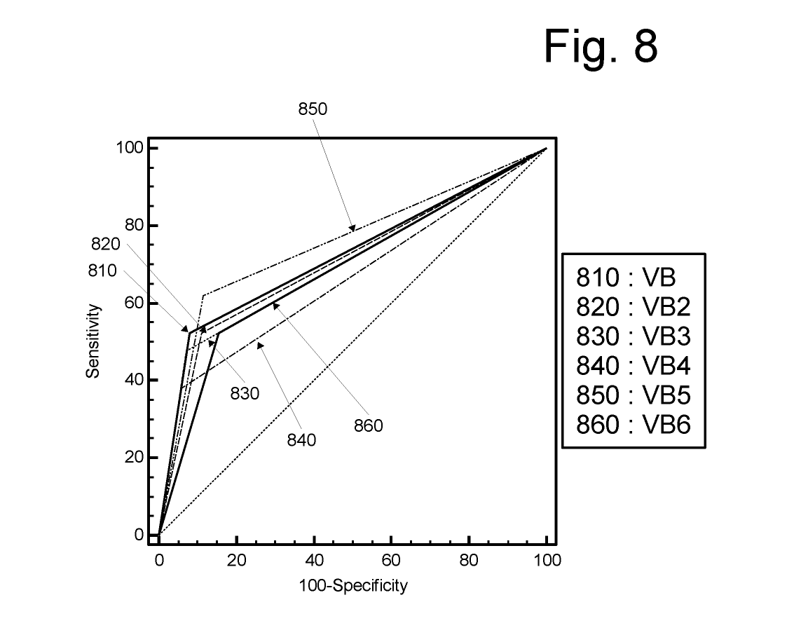

Table 10 shows a sample of cutoff values used in a test of the index's performance in predicting VBD subjects. Each test values are identified by an identifier (VB, VB2, VB3, VB4, VB5 and VB6) and are used in formula (2) (see above) to compute the index. The ROC analyses are preformed and results are shown in FIG. 8. The study was carried out on a cohort of 386 subjects, 21 of which were carriers of VBD.

TABLE 10

| ID | TK1 |
|---|---|
| VB | >=6.5 |
| VB2 | >=5.5 |
| VB3 | >=6.5 |
| VB4 | >=7.5 |
| VB5 | >=6.5 |
| VB6 | >=4.5 |

FIG. 8 is a plot showing the relationship between the sensitivity and the specificity of index computation using cutoff values in a range of values for both TK1 and CRP. The results as characterized by the area under the curve (AUC), the standard error (SE) and the 95% confidence interval (95% CI, are shown in Table 11.

TABLE 11

| | AUC | SE | 95% CI |
|---|---|---|---|
| VB | 0.722 | 0.0685 | 0.588 to 0.856 |
| VB2 | 0.706 | 0.0679 | 0.573 to 0.839 |
| VB3 | 0.702 | 0.0699 | 0.565 to 0.840 |
| VB4 | 0.662 | 0.0716 | 0.521 to 0.802 |
| VB5 | 0.752 | 0.0644 | 0.626 to 0.878 |
| VB6 | 0.684 | 0.0672 | 0.552 to 0.816 |

As can be shown in Table 11 and FIG. 8, TK1 and CPR values produced comparable results in specific ranges, however when reaching some value, e.g., in VB4 at an upper limit for TK1 and in VB6 at a lower limit for TK1, the performance of the index was affected negatively. For CRP, VB3 appears to be a lower limit and VB5 an upper limit. In VB5 while the ROC AUC was higher, there is a trade-off in lack of specificity as other disease states such as cancer would likely be the cause of the variation in the biomarker's level.

Therefore, the acceptable ranges for TK1 and CRP are: TK1 greater or equal 4.5 to 7.5 U/L, with a preferred value of 6.5 U/L, and CRP lower than or equal 3.0 to 12.0 mg/L, with a preferred value of 4.0 mg/L.

An apparatus implementing the invention may be implemented as a computer system, such as a digital computer having a central processing unit, a computer memory, a permanent storage system, and provided with communications interfaces. The communication interfaces comprise means for capturing data, such as an electronic interface that communicates with blood analysis machines, user communication means for receiving input from a user and any other interface means that allow the apparatus to receive data for the purpose of carrying out the method steps of an embodiment of the invention. The apparatus comprises interface means for producing an output such display means, printing means or any other data communication or control means that enable a user to use the result of the invention.

The method steps of the invention may be implemented in a computer program product configured to receive input data (e.g., biomarker data and health status data etc.), and determine ranges for a particular diagnosis. The computer program product may be configured to execute the steps with the apparatus as described above, or within any other computer system that may receive the input for a particular patient, compute the index value and output the result of the diagnosis. The system may stand alone or be embedded in any diagnosis machine.

Thus, a method and apparatus for screening for VBD using the measuring of thymidine kinase activity in a body fluid combined with the concentration of CRP. The invention provides an index that allows a practitioner to determine a probability that a patient is likely a carrier of VBD.

The claimed invention is:

1. A method for screening canine subjects for vector-borne diseases comprising the steps of:
obtaining a serum portion of a blood sample from a canine subject;
obtaining the activity level of thymidine kinase in said serum portion, and determining that thymidine kinase level is greater than 6.5 Unit per liter;
obtaining the concentration of c-reactive protein in said serum portion and determining that the concentration is less than 7 mg per liter; and
determining that said canine subject has a high probability of being affected by a vector-borne disease if said thymidine kinase level is greater than 6.5 Unit per liter and said the concentration of c-reactive protein is less than 7 mg per liter.

2. The method of claim 1 further comprising:
obtaining a thymidine kinase digitized value for said activity level of thymidine kinase, wherein said thymidine kinase digitized value is zero (0) if the activity of thymidine kinase is less than 6.5 U/l, said thymidine kinase digitized value is one (1) if the activity of thymidine kinase is greater than 6.5 U/l and lower than 19.9 U/l, and said thymidine kinase digitized value is two (2) if the activity of thymidine kinase is greater than 20 U/l; and
obtaining a c-reactive protein digitized value for said concentration of c-reactive protein, wherein said c-reactive protein digitized value is zero (0) if the activity of thymidine kinase is less than 6.5 U/l or otherwise said concentration of c-reactive protein is lower or equal to 7 mg/l, said c-reactive protein digitized value is one (1) if said concentration of c-reactive protein is lower or equal to 6.9 mg/l and greater or equal to 4.1 mg/l, and said c-reactive protein digitized value is two (2) if said concentration of c-reactive protein is lower or equal to 4.1 mg/l.

3. The method of claim 2, further comprising:
obtaining a thymidine kinase product value by multiplying said thymidine kinase digitized value by a weighing coefficient of 1.8;
obtaining a c-reactive protein product value by multiplying said c-reactive protein digitized value by a weighing coefficient of 3;
obtaining a vector-borne disease index value by adding said thymidine kinase product value and said c-reactive protein product value; and
determining that said canine subject has a high probability of being affected by a vector-borne disease if said vector-borne disease index is greater than zero (0).

4. The method of claim 1 further comprising:
obtaining a thymidine kinase digitized value for said activity level of thymidine kinase, wherein said thymidine kinase digitized value is zero (0) if the activity of thymidine kinase is less than 6.5 U/l, said thymidine kinase digitized value is one (1) if the activity of thymidine kinase is greater or equal to 6.5 U/l; and
obtaining a c-reactive protein digitized value for said concentration of c-reactive protein, wherein said c-reactive protein digitized value is zero (0) if said concentration of c-reactive protein is lower or equal to 4 mg/l, said c-reactive protein digitized value is one (1) if said concentration of c-reactive protein is greater or equal to 4.1 mg/l;
obtaining a vector-borne disease index value by adding said thymidine kinase digitized value and said c-reactive protein digitized value; and
determining that said canine subject has a high probability of being affected by a vector-borne disease if said vector-borne disease index is greater than zero (0).

5. A system for screening canine subjects for vector-borne diseases comprising:
means for analyzing a serum portion of a blood sample from a canine subject;
means for measuring the activity level of thymidine kinase in said serum portion, and determining that thymidine kinase level is greater than 6.5 Unit per liter;
means for measuring the concentration of c-reactive protein in said serum portion and determining that the concentration is less than 7 mg per liter; and
means for determining that said canine subject has a high probability of being affected by a vector-borne disease if said thymidine kinase level is greater than 6.5 Unit per liter and said the concentration of c-reactive protein is less than 7 mg per liter.

6. The system of claim 5 further comprising:
means for computing a thymidine kinase digitized value for said activity level of thymidine kinase, wherein said thymidine kinase digitized value is zero (0) if the activity of thymidine kinase is less than 6.5 U/l, said thymidine kinase digitized value is one (1) if the activity of thymidine kinase is greater than 6.5 U/l and lower than 19.9 U/l, and said thymidine kinase digitized value is two (2) if the activity of thymidine kinase is greater than 20 U/l; and
means for computing a c-reactive protein digitized value for said concentration of c-reactive protein, wherein said c-reactive protein digitized value is zero (0) if the activity of thymidine kinase is less than 6.5 U/l or otherwise said concentration of c-reactive protein is lower or equal to 7 mg/l, said c-reactive protein digitized value is one (1) if said concentration of c-reactive protein is lower or equal to 6.9 mg/l and greater or equal to 4.1 mg/l, and said c-reactive protein digitized value is two (2) if said concentration of c-reactive protein is lower or equal to 4.1 mg/l.

7. The system of claim 6, further comprising:
means for computing a thymidine kinase product value by multiplying said thymidine kinase digitized value by a weighing coefficient of 1.8;
means for computing a c-reactive protein product value by multiplying said c-reactive protein digitized value by a weighing coefficient of 3;
means for computing a vector-borne disease index value by adding said thymidine kinase product value and said c-reactive protein product value; and
means for determining that said canine subject has a high probability of being affected by a vector-borne disease if said vector-borne disease index is greater than zero (0).

8. The system of claim 5 further comprising:
means for computing a thymidine kinase digitized value for said activity level of thymidine kinase, wherein said thymidine kinase digitized value is zero (0) if the activity of thymidine kinase is less than 6.5 U/l, said thymidine kinase digitized value is one (1) if the activity of thymidine kinase is greater or equal to 6.5 U/l; and
means for computing a c-reactive protein digitized value for said concentration of c-reactive protein, wherein said c-reactive protein digitized value is zero (0) if said concentration of c-reactive protein is lower or equal to 4 mg/l, said c-reactive protein digitized value is one (1) if said concentration of c-reactive protein is greater or equal to 4.1 mg/l;
means for computing a vector-borne disease index value by adding said thymidine kinase digitized value and said c-reactive protein digitized value; and
means for determining that said canine subject has a high probability of being affected by a vector-borne disease if said vector-borne disease index is greater than zero (0).

9. A method for screening canine subjects for vector-borne diseases comprising the steps of:
obtaining a serum portion of a blood sample from a canine subject;
obtaining the activity level of thymidine kinase in said serum portion, and determining that the enzymatic activity of thymidine kinase type 1 is greater than 6.5 Unit per liter; and
determining that said canine subject has a high probability of being affected by a vector-borne disease if said thymidine kinase level is greater than 6.5 Unit per liter.
